(19) United States
(12) Patent Application Publication
(10) Pub. No.: US 2005/0187487 A1
(43) Pub. Date: Aug. 25, 2005

(54) Microsensor catheter and method for making the same

(76) Inventors: Richard G. Azizkhan, Cleves, OH (US); Ian Papautsky, Mason, OH (US); Chong H. Ahn, Cincinnati, OH (US)

Correspondence Address: TAFT, STETTINIUS & HOLLISTER LLP, SUITE 1800, 425 WALNUT STREET, CINCINNATI, OH, 45202-3957, US

(21) Appl. No.: 11/041,906

(22) Filed: Jan. 24, 2005

Related U.S. Application Data
(60) Provisional application No. 60/538,549, filed on Jan. 23, 2004.

(51) Int. Cl. A61B 5/00; A61B 5/02; A61M 25/16; A61M 25/18

(52) U.S. Cl. 600/561; 600/486; 600/549; 600/365

(57) ABSTRACT

A catheter for insertion into a body cavity, duct, or vessel for diagnostic purposes, the catheter including a flexible conduit having a microsensor device mounted thereto that generates output data from a sensed condition. The microsensor device is operatively coupled to a data acquisition device to communicate the output data to the data acquisition device. The microsensor device is mounted to a flexible dielectric substrate carrying a lead, and the flexible dielectric substrate is separate from and carried by the flexible conduit.

### CROSS-REFERENCE TO RELATED APPLICATION

This application claims priority to and benefit of Provisional Application No. 60/538,549, filed on Jan. 23, 2004, which is herein incorporated by reference in its entirety.

### BACKGROUND

1. Field of the Invention

The present invention is directed to biomedical technology intended for enhancing the care and treatment of patients, and more specifically to a physiologic monitor or catheter that may be utilized in the diagnosis and/or treatment of neonatal patients by transmitting real-time information to a data acquisition device.

2. Background of the Invention

Intravascular blood pressure measurements in patients are most commonly performed using a saline filled pressure transferring catheter and external pressure sensors. By measuring the pressure with a miniature sensor inside the artery, time response and accuracy can be improved.

There are a number of approaches used to measure pressure and flow with microscale sensors. Pressure sensors represent a fairly mature and commercially available area of Microelectromechanical Systems (MEMS) sensors. Most commonly, microscale pressure sensors use either capacitive or piezoresistive measurement schemes. Capacitive pressure sensors measure change in capacitance between two parallel plates, while one plate deflects due to pressure changes. Small solid-state capacitive pressure sensors are typically non-linear but normally have the advantage of high pressure sensitivity and low temperature drift. Piezoresistive sensors rely on the piezoresistive effect in which sensor resistance changes in proportion to changes of sensor dimensions, which might result from changes in pressure. A piezoresistive detection technique may be more favorable than capacitive detection technique for miniaturized sensors due to better scaling characteristics. An important advantage of piezoresistive sensors is that the amplifier circuitry can be placed far away from the sensors, e.g., outside the body.

Flow measurements are most commonly performed using a well-known “hot-wire” anemometer principle. In this case, fluid is locally heated to a few degrees above ambient, and the temperature change is measured upstream and downstream of the heater. The temperature varies proportionally with the flow, and thus flow can be calculated though knowledge of the temperature coefficient of resistance of the heater and a simple electrical measurement of its resistance. An alternative technique for measuring flow velocities is thermal tracing in which a heater is pulsed and the “time of flight” of the heated fluid is determined using a downstream temperature sensor.

Biomedical devices have been used in the clinical setting for quite some time. From the development of the simple blood-pressure cuff to the development of neural network devices for adjusting a neonate's ventilator setting, biomedical devices are instrumental to the delivery of care. Recent developments in MEMS sensors suggest that the quality of pediatric care can be significantly enhanced once these advancements are translated into the pediatric setting. For example, MEMS sensors can capture and report various environmental characteristics, i.e., flow rate, temperature, crack detection, and concentration.

Every year in the United States there are hundreds of thousands of premature and term neonates with significant disease states that include respiratory distress syndrome, cardiac disease, and a variety of surgical disorders requiring intensive care. The current monitoring capacity and capabilities are very crude for this group of patients. Attending physicians primarily rely on physical signs, examination and relatively crude instrumentation. Current methodology to measure tissue perfusion, blood flow and pressures are extremely primitive and cumbersome. Those patients that have indwelling arterial catheters may risk significant complications associated with the placement of these monitoring devices. In addition, the volume of blood drawn from children for tests represents a significantly higher percentage of the total blood volume than the same volume if drawn from an adult. Such blood tests may include testing for glucose and electrolyte concentrations.

Neonates requiring critical care are often transfused on a regular basis since blood drawn for tests may induce anemia. Minimizing the volume of blood drawn and new blood banking techniques have reduced the transfusion requirement and risks. However, new monitoring paradigms using the MEMS technology could drastically reduce transfusion requirements and enhance diagnostic speed and treatment.

### SUMMARY OF THE INVENTION

The present invention is directed to biomedical technology intended for enhancing the care and treatment of patients, and more specifically to a physiologic monitor or catheter that may be utilized in the diagnosis and/or treatment of neonatal patients by transmitting real-time information to a data acquisition device.

Exemplary embodiments of the present invention utilize microsensor devices to detect environmental conditions, most notably physiological conditions of a patient, and convey the data generated indicative of these detected environmental conditions to a display or a data acquisition device, such as, without limitation, a computer. Most often, the microsensor devices are mounted to the interior of a catheter or a subcomponent of a catheter such as a tube, and are oriented to enable a sensor element of the microsensor to interact with the external environment. Interaction with the external environment may include indirect interaction as well as direct interaction. Each microsensor device may relay data to the display or data acquisition device indicative of the environmental conditions detected in real-time. Exemplary applications of such an embodiment include a catheter having a plurality of microsensor devices that detect a plurality of differing environmental conditions, where the catheter is dimensioned to facilitate application as a neonatal diagnosis and/or treatment device.

Each microsensor device may be mounted to a flexible substrate prior to being mounted to or within a catheter or a tube. In exemplary embodiments, the flexible substrate is in the form of an elongated strip or ribbon of dielectric materal, where the microsensor device is mounted approximate the leading end of the strip. While the present invention is discussed herein as utilizing an exemplary flip-chip bonding technique, the use of such technique is not required, as those of ordinary skill, particularly in the semiconductor fields, are familiar with means for mounting a chip onto conductive leads. The flexible dielectric substrate includes electrical leads for bonding to the microsensor device, and may also include electrical leads connected to the display and/or data acquisition device. While such leads may be coupled to the display and/or data acquisition device, it is also envisioned that such microsensor devices communicate wirelessly with the display or data acquisition device using appropriate RF components. By mounting the microsensor devices to the flexible dielectric substrate, the combination of such eases the production capabilities and lessens the difficulties of mounting such microsensor devices within the catheter or tube.

It is thus a first aspect of the present invention to provide a catheter for insertion into a body cavity, duct, or vessel for diagnostic purposes, where the catheter includes a flexible conduit having a microsensor device mounted thereto that generates output data from a sensed condition, where the microsensor device is operatively coupled to a data acquisition device to communicate the output data to the data acquisition device, where the microsensor device is mounted to a flexible substrate carrying a lead, and where the flexible substrate is separate from and carried by the flexible conduit.

In a more detailed embodiment of the first aspect, the flexible conduit is oriented in parallel with a second conduit. In yet another more detailed embodiment, the flexible conduit includes an orifice therein to expose a sensor element of the microsensor device to a sensed condition external from the flexible conduit. In a further detailed embodiment, the flexible conduit is circumscribed by a larger conduit having an opening therein to align with the orifice of the flexible conduit to expose the sensor element to the sensed condition external from the larger conduit. In still a further detailed embodiment, the larger conduit circumscribes a tube oriented in parallel with the flexible conduit, where the tube includes a distal end having an aperture therein and a proximal end having an opposed aperture therein, and where the tube also inhibits fluid communication between an interior of the tube and an interior of the flexible conduit. In a more detailed embodiment, the interior of the flexible conduit includes the microsensor device, the flexible substrate, and the lead running between the microsensor device and the data acquisition device. In a more detailed embodiment, the sensor element may detect glucose concentration, pressure, temperature, pH, oxygen concentration, carbon dioxide concentration, heartbeat, water or other fluids, the presence of certain chemicals and/or the presence of certain biologic materials. In yet another more detailed embodiment of the first aspect, the flexible conduit circumscribes a tube including a distal end having an aperture therein and a proximal end having an opposed aperture therein, where the tube also inhibits fluid communication between an interior of the tube and an interior of the flexible conduit housing the microsensor device, the flexible dielectric substrate, and the lead running between the microsensor device and the data acquisition device.

In an alternate detailed embodiment of the first aspect, the catheter includes a protective coating over at least the flexible substrate, the first electrically conductive material and the microsensor device. In yet a further detailed embodiment the protective coating includes a layer of a poly dimethyl siloxane (PDMS) material.

It is a second aspect of the present invention to provide a catheter for insertion into a body cavity, duct, or vessel for diagnostic purposes, where the catheter includes a flexible conduit carrying at least two microsensor devices therewith, each of which respectively generate output data from respective sensed conditions, where each microsensor device is operatively coupled to a data acquisition device to communicate the output data to the data acquisition device, where at least the two microsensor devices are mounted to a flexible dielectric substrate carrying a lead, and where the flexible dielectric substrate is separate from and carried by the flexible conduit.

In a more detailed embodiment of the second aspect, the flexible conduit is oriented in parallel with a second conduit. In still another more detailed embodiment, the flexible conduit includes at least one orifice therein to respectively expose a sensor element of at least one of the two microsensor devices to the respective sensed condition external to the flexible conduit. In a further detailed embodiment, the flexible conduit is circumscribed by a larger conduit having at least two openings therein to align with at least the two orifices of the flexible conduit to respectively expose at least two sensor elements of the at least two microsensor devices to the respective sensed conditions external from the larger conduit. In still a further detailed embodiment, the larger conduit circumscribes a tube oriented in parallel with the flexible conduit, where the tube includes a distal end having an aperture therein and a proximal end having an opposed aperture therein, and where the tube also inhibits fluid communication between an interior of the tube and an interior of the flexible conduit. In a more detailed embodiment, the interior of the flexible conduit includes the at least two microsensor devices, the flexible dielectric substrate, and the lead running between the at least two microsensor devices and the data acquisition device. In a more detailed embodiment, at least one of the at least two sensor elements of the at least two microsensor devices may detect glucose concentration, pressure, temperature, pH, oxygen concentration, carbon dioxide concentration, heartbeat, water or other fluids, the presence of certain chemicals and/or the presence of certain biologic materials; and another of the at least two sensor elements of the at least two microsensor devices also may detects glucose concentration, pressure, temperature, pH, oxygen concentration, carbon dioxide concentration, heartbeat, water or other fluids, the presence of certain chemicals and/or the presence of certain biologic materials. In another more detailed embodiment, the flexible conduit circumscribes a tube including a distal end having an aperture therein and a proximal end having an opposed aperture therein, where the tube also inhibits fluid communication between an interior of the tube and an interior of the flexible conduit housing the flexible dielectric substrate and the at least two microsensor devices. In yet another more detailed embodiment, at least two of the microsensor devices are mounted to the flexible dielectric substrate utilizing a flip-chip bonding technique. In yet a further more detailed embodiment, the at least two microsensor devices are proportionally spaced along at least a part of the length of the flexible conduit.

It is a third aspect of the present invention to provide a method of coupling a microsensor device onto a conduit adapted for use as a component of a catheter. The method includes the step of: (a) providing a flexible dielectric substrate having a leading end; (b) applying a first electrically conductive material to the dielectric substrate approximate the leading end to provide at least one lead; (c) coupling the first electrically conductive material to a microsensor device to provide electrical communication between the microsensor device and the at least one lead; and (d) mounting the leading end of the dielectric substrate to a conduit of a catheter.

In a more detailed embodiment of the third aspect, the method further comprises the step of laser cutting the flexible dielectric substrate to separate deposits of the first electrically conductive material. In yet another more detailed embodiment, the method further comprises the steps of: (e) applying a photoresist material onto the flexible dielectric substrate; (f) selectively developing the photoresist material; and (g) selectively removing undeveloped photoresist material to form a cavity bounded in part by the first electrically conductive material.

In an alternate detailed embodiment of the third aspect, the coupling step includes the steps of applying a second electrically conductive material onto a portion of the first electrically conductive material, and coupling the second electrically conductive material to a microsensor device. In a further detailed embodiment, the first electrically conductive material comprises a metal or metal alloy. In yet a further detailed embodiment, the second electrically conductive material includes a conductive polymer material. In still a further detailed embodiment, the coupling step includes flip-chip bonding of the microsensor device to the second electrically conductive material.

In a more detailed embodiment of the third aspect, the method further includes the steps of: (h) providing a substrate carrier; and (i) removing the substrate carrier from the flexible dielectric substrate prior to the coupling step, where the removing step is accomplished at least in part by peeling the substrate carrier from the flexible dielectric substrate. In still another more detailed embodiment, the substrate carrier comprises a silicon wafer. In a further detailed embodiment, the coupling step includes the step of applying an alignment pedestal to the flexible dielectric substrate to align the microsensor device with respect to the flexible dielectric substrate.

In more detailed embodiment of the third aspect, the method further comprises the step of coating at least the dielectric substrate, the first electrically conductive material and the microsensor device with a protective material. In yet a further detailed embodiment the protective material includes a layer of a poly dimethyl siloxane (PDMS) material.

It is a fourth aspect of the present invention to provide a multilumen catheter that includes: (a) a first flexible tube having a microsensor device at least partially carried thereon and operatively coupled to an output device, where the microsensor device is mounted to a flexible dielectric substrate carrying a lead isolated from an environment external to the first flexible tube, and where the microsensor device has a sensor element exposed to the environment to detect environmental conditions in real-time and relay data evidencing such environmental conditions to the output device in real-time; (b) a second flexible tube including a distal end having an orifice therein and providing a conduit therein; and (c) a catheter receiving at least the first flexible tube and the second flexible tube.

In a more detailed embodiment of the fourth aspect, the catheter includes a wall circumscribing the first flexible tube and the second flexible tube substantially along the length of the first flexible tube and the second flexible tube. In yet another more detailed embodiment, the flexible dielectric substrate and/or the lead is encased by the first flexible tube. In a further detailed embodiment, the first flexible tube has a diameter of between about 0.67 mm to about 1.67 mm. In still a further detailed embodiment, the second flexible tube has a diameter of between about 0.33 mm to about 2 mm.

In a more detailed embodiment of the fourth aspect, the first flexible tube includes an aperture to provide the sensor element with access to the environment. In still another more detailed embodiment, the catheter wall includes an opening generally aligned with the aperture of the first flexible tube to provide the sensor element with access to environmental conditions beyond the catheter wall. In a further detailed embodiment, the first flexible tube is coaxial with the second flexible tube, and the second flexible tube includes an aperture generally aligned with the aperture of the first flexible tube to provide the sensor element with access to the external environment.

It is a fifth aspect of the present invention to provide a multilumen catheter that includes: (a) a first flexible tube having a first microsensor device carried thereon, where the first microsensor device is coupled to a first flexible dielectric substrate carrying a set of first conductive leads in electrical communication with the first microsensor device, where the first flexible tube provides a conduit though which the first flexible dielectric substrate is received, and where the first microsensor device includes a sensor element in communication with an environment external to the first flexible tube to detect an environmental condition in real-time and relay data evidencing such environmental condition in real-time to a remote data processing device; (b) a second flexible tube having a second microsensor device carried thereon, where the second microsensor device is coupled to a second flexible dielectric substrate carrying a second set of conductive leads in electrical communication with the second microsensor device, where the second flexible tube provides a passageway though which the second flexible dielectric substrate is received, and where the second microsensor device includes a sensor element in communication with an environment external to the second flexible tube to detect an environmental condition external to the second flexible tube in real-time and relay data evidencing such environmental condition in real-time to the remote data processing device; and (c) a third flexible tube including a distal end having an orifice therein and providing a conduit for fluid flow therethrough

### BRIEF DESCRIPTION OF THE DRAWINGS

FIG. 1A-1G are cross-sectional views of exemplary process stages for mounting a microsensor device onto a flexible substrate in accordance with an exemplary embodiment the present invention;

FIG. 2 is a segmental view of an exemplary dual tube catheter in accordance with an exemplary embodiment of the present invention within a bodily cavity;

FIG. 3 is a end view of an exemplary single tube catheter in accordance with an exemplary embodiment of the present invention; and

FIG. 4 is a cross-sectional view of the single tube catheter of FIG. 3 in accordance with an exemplary embodiment the present invention;

FIG. 5 is an end perspective segment view of the single tube catheter of FIG. 3 in accordance with an exemplary embodiment of the present invention;

FIG. 6 is a segmental view of another exemplary single tube catheter in accordance with an exemplary embodiment of the present invention within a bodily cavity;

FIG. 7 is a segmental view of another dual tube catheter in accordance with an exemplary embodiment the present invention within a bodily cavity;

FIGS. 8A-8F are perspective views of exemplary process stages for mounting a microsensor device onto a flexible substrate in accordance with an exemplary embodiment of the present invention; and

FIG. 9 is a schematic diagram representing a testing platform for testing the exemplary embodiment fabricated according to stages shown in FIGS. 8A-8F.

### DETAILED DESCRIPTION

The exemplary embodiments of the present invention are described and illustrated below to encompass methods of mounting microsensors onto a flexible substrate, and catheters incorporating such microsensors mounted to a flexible substrate adapted to be utilized as a neonatal diagnostic apparatus. Of course, it will be apparent to those of ordinary skill in the art that the preferred embodiments discussed below are exemplary in nature and may be reconfigured without departing from the scope and spirit of the present invention. However, for clarity and precision, the exemplary embodiments as discussed below may include optional steps and/or features that one of ordinary skill should recognize as not being a requisite to fall within the scope and spirit of the present invention.

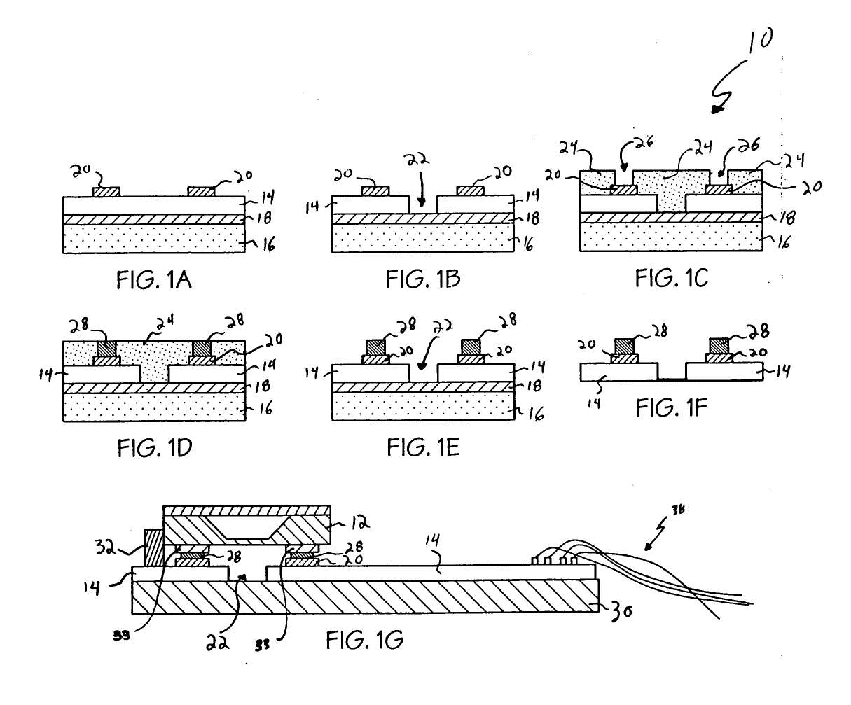

Referencing FIGS. 1A-1G, an exemplary flip-chip bonding process 10 will be described for mounting a microsensor device 12 onto a substrate 14 prior to the substrate being mounted to a catheter or a tube of a catheter (See FIGS. 2-7). Referring to FIG. 1A, the exemplary process may begin with a silicon wafer 16 having a layer of poly dimethyl siloxane (PDMS) 18 deposited thereon collectively acting as a base for multiple layer deposition. The PDMS 18 acts as a bonding layer onto which the substrate 14 is layered. In this exemplary process 10, the substrate 14 utilized for purposes of explanation is a polyimide film commercially available as Kapton® from E.I. du Pont de Nemours and Company, Inc. Conductive leads 20 are formed onto the substrate 14 by utilization of a photoresist to define a cavity into which a chromium layer and a gold layer are deposited. A more detailed discussion of the process and functionality of a photoresist is discussed below. It should be understood that it is not required that the substrate 14 be mounted to the PDMS 18 prior to deposition of the conductive leads 20. For purposes of explanation in the present embodiment, the conductive leads 20 comprise a layer of chromium and a layer of gold, however, or other conductive material(s) sufficient to provide the functionality associated with a conductive lead may be utilized and concurrently fall within the scope of the invention.

Referring to FIGS. 1B and 1C, a laser cutting technique is utilized to form a notch 22 within the substrate 14 extending down to the PDMS layer 18 between the conductive leads 20. As will be discussed below, this notch 22 provides an aperture for allowing a sensing element (not shown) to access an environment external to a catheter to which the substrate 14 will be bonded. A photoresist material 24 (such as AZ4620) is then spin coated onto the substrate section 14 to concurrently cover the substrate 14, the notch 22, and the conductive leads 20. A pattern (not shown) is positioned over the photoresist material 24 having openings therethrough for ultraviolet light to shine and develop selected segments of the photoresist material. In regions where the photoresist material is not developed, it may easily be removed (as shown in FIG. 1C) to leave a cavity 26 bounded by the developed photoresist material 24 and the conductive leads 20.

Referencing FIGS. 1D-1F, a conductive material, such as a conductive polymer (Epo-Tek-K/5022-115 BE), is deposited to substantially fill the cavity 26 to form conductive bumps 28 bonding the conductive material to the conductive leads 20. A convection baking process ensues for fifteen minutes at 100° C. Those of ordinary skill in the art will realize that the conductive material 28 may be deposited in a layer onto the developed photoresist 24 to at least substantially fill the cavity 26. Thereafter, the photoresist 24 is removed, leaving the stacked structure shown in FIG. 1E. FIG. 1F reflects removal of the PDMS 18 and the silicon wafer 16 from the substrate 14. Such removal may be accomplished by techniques know to those of ordinary skill, and include, without limitation, separation of the PDMS 18 from the substrate 14 by peeling the substrate 14 from the PDMS 18. Exemplary measurements for the conductive bumps 28 as shown in FIG. 1F include a thickness of about 25 μm and an area of about 80 μm by 250 μm.

Referring to FIG. 1G, the substrate 14 is positioned onto a heating stage 30 where a laser micromachined stop 32 is positioned in proximity to the substrate 14 as an alignment device to align a die carrying the microsensor device 12 prior to mounting the microsensor device 12 to the conductive material 26. Exemplary microsensor devices include piezoresistive pressure sensing chips (such as SM5106) commercially available from Silicon Microstructures, Inc. It is to be understood that the stop 32 is not necessary in carrying out the alignment and bonding of the pressure microsensor device 12 to the conductive material 28. In sum, aluminum electrical contacts 33 of the microsensor device 12 are mounted to the conductive material 28 utilizing a flip-chip bonding technique carried out at 170° C. such that the conductive material 28 melts to provide electrical communication between the microsensor device 12 contacts 33 and the conductive material 28. An ultraviolet adhesive (not shown) may be applied around the microsensor device 12 to provide additional bonding strength. Conductive wires 38, rated at 38 AWG, are bonded to the conductive leads 20 using a silver epoxy and optionally covered with an ultraviolet adhesive as a protective layer. Thereafter, laser micromachining is utilized to cut the substrate 14 to an elongated strip sized for insertion into a catheter or a tube thereof.

The flip-chip bonding using a conductive polymer instead of solder has several advantages, such as simplicity of processing, high bumping alignment resolution (<5 μm), and a lower bonding temperature (approx. 170° C.). The compliant polymer bumps exhibit improvement in the tolerance to thermal stress caused by mismatched thermal expansion coefficients of the chip, the substrate, and the passivation layer. These characteristics enable the use of polymer flip-chip bonding technique with the polymer substrates, which may not be able to be processed at high temperatures needed for the traditional solder flip-chip bonding. Nevetherless, it is within the scope of certain aspects of the present invention to utilize either polymer flip-chip bonding or solder flip-chip bonding.

Referencing FIG. 2, an exemplary dual tube catheter 40 is shown in a segmental view within a blood vessel 42 of a patient, for example. The catheter 40 includes a circumferential wall 44 that circumscribes a first tube 46 adjacent to a second tube 48 in a parallel or stacked configuration. This orientation provides three generally distinct lumens within the catheter 40: a first lumen 50 bounded by an interior surface 52 of a cylindrical wall 54 of the first tube 46; a second lumen 56 bounded by an interior surface 58 of a cylindrical wall 60 of the second tube 48; and, a third lumen 59 bounded by an interior surface 62 of the circumferential wall 44 of the catheter 40 and exterior surfaces 64, 66 of the cylindrical walls 54, 60 of the first and second tubes 46, 48.

The first tube 46 includes an aperture 68 aligned with a sensing element 69 of a sensor device 70, carried on a flexible substrate 76 that is mounted to the interior surface 52 the cylindrical wall 54. The sensing element 69 and opening 68 are likewise aligned with an aperture 72 in the circumferential wall 44 of the catheter 40. Thus, the apertures 68, 72 allow communication between the sensing element 69 and an environment external 74 to the catheter 40. The first lumen 50 houses the sensor device 70 that is carried on a leading end of a flexible substrate 76, such as Kapton®, as described above. The flexible substrate 76 includes conductive leads 80 mounted thereto for providing electrical communication between conductive bumps (not shown) mounted to the sensor device 70, and a series of wire leads 82 mounted at an opposing end of the substrate 76. A power source and/or a digital processor (not shown) for processing data received from the sensor device 70 may be coupled or operatively coupled to the wire leads 82.

While the flexible substrate 76 is shown as extending through a portion of the first tube 46, it is within the scope of the invention to provide a flexible substrate having a length substantially equaling that of the first tube 46. It is also within the scope of the invention to provide shortened sections of the flexible substrate where the wire leads 82 are fashioned to have a length substantially equaling the length of the first tube 46. The wires 82 may be longer than the first tube 46 and can connect directly to an A/D system or via a miniature connector and a shielded cable. It is further within the scope of the invention that the length of the first tube be longer or shorter than the length of the second tube.

The second lumen 56 of the second tube 48 is intended to accommodate the functionality associated with a typical catheter to include, without limitation, draining a fluid from a particular area of the body or administering a fluid to a particular area of the body. While the second lumen 56 is shown as being fluidically separated from the first and third lumens 50, 58, it is within the scope of the invention for the first lumen 50 to communicate with the second lumen 56.

An exemplary configuration might include an opening within the cylindrical wall 60 of the second tube 48 in alignment with a corresponding orifice within the cylindrical wall 54 of the first tube 46. Concurrently, a sensor might be mounted in alignment with the corresponding orifice to provide communication between the sensor and the second lumen 56. Such an embodiment might be utilized for measuring for example, without limitation, the pH, pressure, flow rate, temperature, or other relevant information regarding the environment within the second lumen.

To mount the flexible substrate 76 and the sensor device 70 within the catheter 40, an adhesive, for example, 3321 (from a product line having different viscosities represented by 33XX), a UV-cured medical grade adhesive from Loctite®, is applied to the sensor device 70 prior to insertion of the sensor device 70 and flexible substrate 76 within the first tube 46. The sensor device 70 is positioned within the first tube 46 so as to align the sensing element 69 with the aperture 68. Thereafter, the first tube 46 is exposed to UV light to cure the adhesive and mount the sensor device 70 to the tube 46. A further exemplary adhesive is LC-1111 (from a product line including product numbers LC-1110 thru LC-1113 and LC-1210 thru LC-1214) from 3M®. Such an exemplary procedure for utilizing the adhesive would encompass applying LC-1111 to the sensor device 70 and thereafter aligning the sensor device 70 within the tube 46 such that the aperture 68 and sensing element 69 are aligned. The adhesive is thereafter exposed to UV-light sufficient to cure the adhesive and mount the sensor device 70 to the tube 46. As discussed above, the sensor device 70 may be aligned within the first tube 46 prior to fabrication of the aperture 68 within the tube 46.

Exemplary materials that may be utilized in whole or in part as the flexible substrate generally include biocompatible flexible polyurethanes, polyimides, or silicones. Biocompatible generally refers to the feature of a material to inhibit harmful chemicals from leaching out. Such exemplary materials may be electrically insulating, resistant to chemicals used to pattern metallic traces thereon, and thermally stable above 150° C.

Exemplary measurements for the catheter 40 may include a diameter of less than 1.7 mm. Exemplary measurements for the sensor devices 70 may include a limiting dimension of less than 1.7 mm. Exemplary sensor devices 70 may have a limiting dimension of less than 0.7 mm. Exemplary measurements for the catheter 40 include a diameter of between about 0.67 mm to about 1.67 mm. Exemplary measurements for the first tube 46 include a diameter of between about 0.33 mm to about 2 mm.

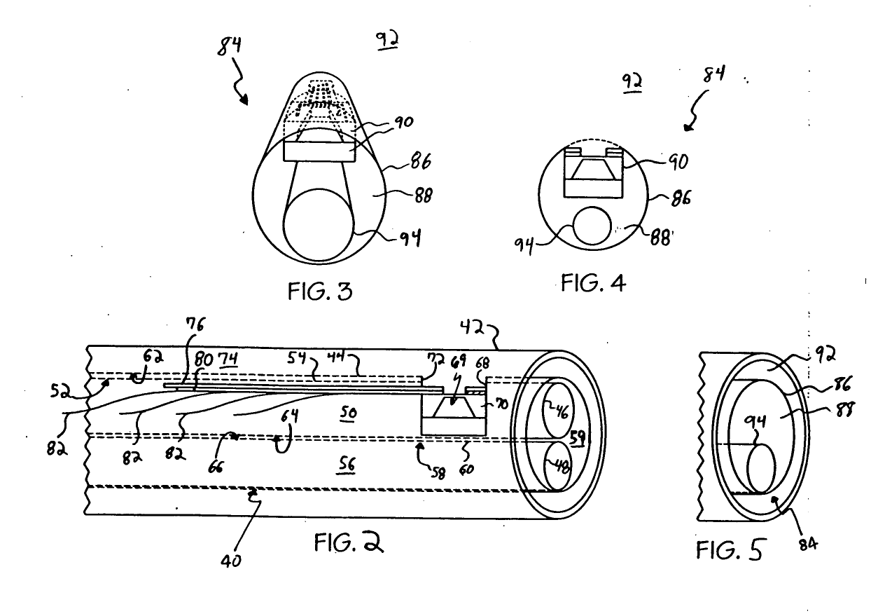

Referring to FIGS. 3, 4, and 5, an exemplary single tube catheter 84 includes an outer circumferential wall 86 defining an internal conduit 88 housing at least one sensor 90. As shown in FIG. 3, a portion of the circumferential wall 86 has been removed to provide direct communication between the sensor 90 and an environment 92 external to the catheter 84. In this way, the sensor 90 may be configured and/or capable of sensing for example, without limitation, glucose concentration, temperature, pressure, pH, position, etc. Those of ordinary skill are familiar with the conditions of a neonate that may be monitored for diagnostic and/or observatory purposes.

The catheter 84 also includes a tube 94 adapted to accommodate the functionality typically associated with a catheter and/or tube. Such uses have been non-exhaustively discussed above, and those of ordinary skill will readily understand the scope of such uses.

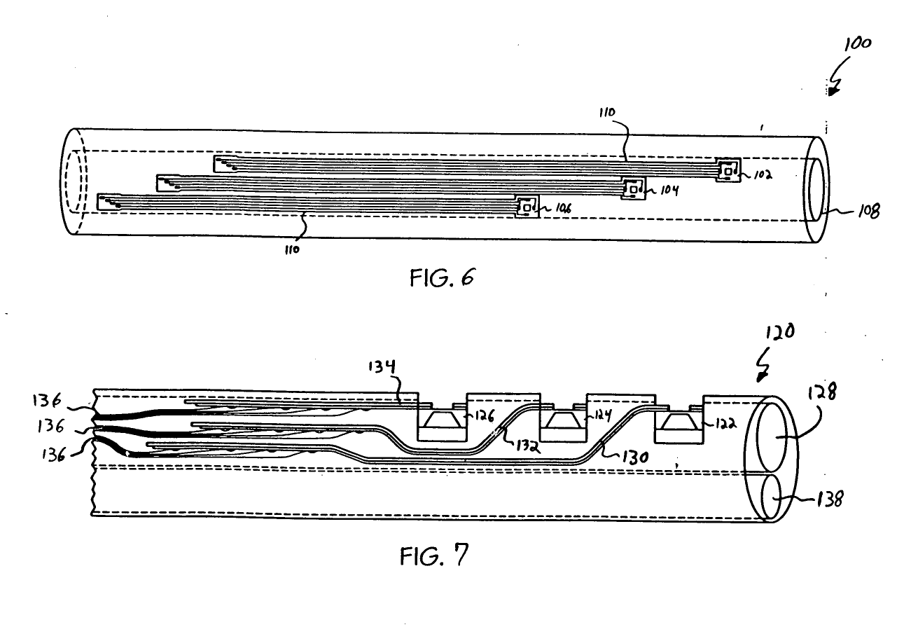

Referencing FIG. 6, an alternate exemplary single tube catheter 100 includes a plurality of sensors 102, 104, 106 mounted to a circumferential wall 108 thereof, providing access to external points along the length of the catheter 100. Such an exemplary embodiment may utilize a flexible substrate 110, as discussed above, along with positioning and mounting techniques discussed above. In such an exemplary embodiment, the sensors 102, 104, 106 may be of the same type to monitor for the same condition, or may conversely be configured to sense more than one condition. For example, the first sensor 102 may be a pressure sensor, while the second sensor 104 might be a temperature sensor, and the third sensor 106 might measure pH or concentration sensor for monitoring, for example, glucose. As discussed above, the sensor data is communicated to a remote device for data capture and/or analyzation.

Referring to FIG. 7, a further alternate exemplary dual tube catheter 120 includes multiple sensors 122, 124, 126 mounted to a first tube 128. Each microsensor device 122, 124, 126 is mounted to a separate flexible substrate 130, 132, 134. As discussed above, each flexible substrate 128, 130, 132 includes conductive leads 134 and wire leads 136 mounted thereto. The wire leads 136 are coupled or operatively coupled to a data recordation or analyzation device (not shown) to provide real time or limited delay information regarding data indicative of microsensor device 122, 124, 126 sensed conditions. Those of ordinary skill will realize that such a sensor might be a temperature sensor, a position sensor, a glucose sensor, a pH sensor, etc., having communicative contact with the requisite environment for sensing one or more conditions. A second tube 138 may be utilized as a conduit for fluid flow or other functionality associated with a catheter.

It is also within the scope and spirit of the present invention to provide a catheter having more than two tubes, where one or more such tubes include at least one microsensor mounted thereto capable of sensing environmental conditions and relaying data indicative of such environmental conditions to a data processing device. Those of ordinary skill are familiar with bundled catheters having three or more tubes bundled together.

As referred to above, a second tube 138 may provide functionality associated with a prior art catheter. Those of ordinary skill are familiar with such uses.

As referred to above, a sensor includes any device capable of providing responsive data indicative of conditions in proximity the sensed area. Exemplary sensors include pressure sensors from Silicon Microstructures, Inc. or MEMS sensors commercially available from Sporian Microsystems (Sporian has developed MEMS sensors for sensing temperature and pressure, for example; and has developed a line of MEMS biosensors for detecting certain chemical and biologic pathogens). Other commercially available microsensors and/or MEMS sensors are commercially available technology; and are available for production by those of ordinary skill.

Another exemplary embodiment of a pressure microsensing catheter for neotatal care has been developed to measure intravascular blood pressure. This embodiment uses polymer flip-chip bonding on a flexible Kapton film for mounting silicon pressure microsensors into French dual lumen neonatal catheters (1.67 mm o.d.). This embodiment was fabricated and tested in a nitrogen pressure chamber and in water in a compressible container.

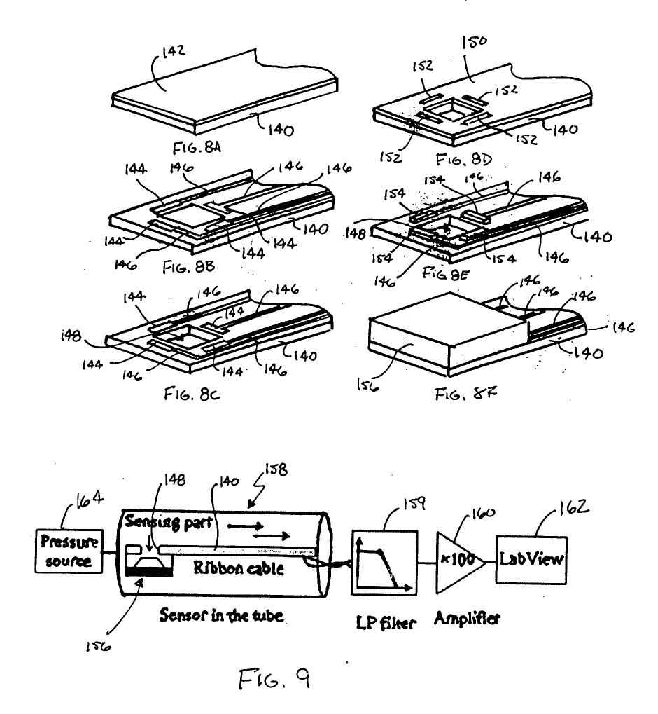

Referring to FIGS. 8A through 8F, the fabrication of this exemplary embodiment utilized flip-chip bonding to bond a commercially available 0-15 psi pressure microsensor to a flexible Kapton film for the dual-lumen neonatal catheter. Referring to FIG. 8A, in an initial fabrication step, Kapton film (approx. 1.5 mm wide and approx. 15 mm long) 140 with a 25-μm thick Cu layer (Dupont) 142 was cleaned. Referring to FIG. 8B, contact pads 144 and wire/lead traces 146 were defined in Cu using photolithography and etching with ferric chloride (Fisher Scientific). Referring to FIG. 8C, a section of the Kapton film covering the sensor membrane was removed by laser micromachining to form a sensing window 148. Referring to FIG. 8D, an AX4620 photoresist (Clariant, Inc.) 150 was deposited as a sacrificial material and patterned by lift-off. The lifted-off pattern defines four openings 152 for depositing the conductive bumps for the mounting the microsensor thereto. Referring to FIG. 8E, the openings were filled with a conductive polymer (Epo-Tek-K/5022-115BE), and then the photoresist was stripped off leaving the four conductive polymer bumps 154. And referring to FIG. 8F, a pressure sensor die 156 was aligned over the polymer bumps on the Kapton film and the sensor was flip-chip bonded at 170° C. The sensor was then inserted into catheter tubing with an opening cut for sensing the area as described and illustrated in previous embodiments. For connection to external electronics lead wires were soldered to the Cu pads. The Kapton film ribbon cable was then coverd with poly dimethyl siloxane (PDMS) as a protective layer. In initial tests the PDMS was cured at 100° C. for one hour forming a relatively thick layer over the sensing area, reducing sensitivity by approximately 15%. In later tests the PDMS was cured for 24 hours, resulting in a significantly thinner coating and only approximately 2% reduction in sensitivity.

The pressure sensors used in the several version of this embodiment were the commercially available SM5106 piezoresistive pressure sensor dies or the commercially available SM5108 piezoresistive pressure sensor dies from Silicon Microstructures, Inc. The SM5106 pressure sensor has dimensions of 1.56 mm×1.56 mm×0.9 mm. The SM5108 pressure sensor has dimensions of 0.65 mm×0.65 mm×0.65 mm.

As shown in FIG. 9, to test this embodiment, the microsensing catheter 158 was coupled to a signal conditioning platform (2345 National Instruments) to reduce noise and amplify the signal. An LP01 (0-100 Hz) low pass filter 159 and a SG04 strain gauge amplifier 160 with a gain of 100 were used for signal conditioning. A computer running LabView software (ver. 7, National Instruments) 162 with a data acquisition board (NI6036E, National Instruments) was used to collect data. The microsensing catheter was tested in a nitrogen pressure chamber and then in water in a compressible container 164. The water container was compressed and released in the range of normal blood pressure (80-120 mm Hg) to simulate blood pressure changes. The tests showed that the sensors performed linearly in the desired pressure range, with typical sensitivity of 6 mmHg/mV. The sensors operated within 0.5 mmHg error over the range of 0-500 mmHg.

Following from the above description and invention summaries, it should be apparent to those of ordinary skill in the art that, while the methods and apparatuses described herein constitute exemplary embodiments of the present invention, the invention is not limited to these precise embodiments and changes may be made to such embodiments without departing from the scope of the invention as defined by the claims. Additionally, it is to be understood that the invention is defined by the claims and it is not intended that any limitation or element describing the exemplary embodiments set forth herein is to be incorporated into the interpretation of any claim element unless such limitation or element is explicitly stated. Likewise, it is to be understood that it is not necessary to meet any or all of the identified advantages or objects of the invention disclosed herein in order to fall within the scope of any claims, as the invention is defined by solely by the claims and since inherent and/or unforeseen advantages of the present invention may exist even though they may not have been explicitly discussed herein.

1. A catheter for insertion into a body cavity, duct, or vessel for diagnostic purposes, the catheter comprising:
a flexible conduit having a microsensor device adapted to generate output data from a sensed condition mounted thereto, the microsensor device being operatively coupled to a data acquisition device to communicate the output data to the data acquisition device, wherein the microsensor device is mounted to a flexible substrate carrying a lead, and wherein the flexible substrate is separate from and carried by the flexible conduit.

2. The catheter of claim 1, wherein the flexible conduit is oriented in parallel with a second conduit.

3. The catheter of claim 2, wherein the flexible conduit includes an orifice therein to expose a sensor element of the microsensor device to a sensed condition external from the flexible conduit.

4. The catheter of claim 3, wherein the flexible conduit is circumscribed by a larger conduit having an opening therein to align with the orifice of the flexible conduit to expose the sensor element to the sensed condition external from the larger conduit.

5. The catheter of claim 4, wherein the larger conduit circumscribes a tube oriented in parallel with the flexible conduit, the tube including a distal end having an aperture therein and a proximal end having an opposed aperture therein, the tube also inhibiting fluid communication between an interior of the tube and an interior of the flexible conduit.

6. The catheter of claim 5, wherein the interior of the flexible conduit includes the microsensor device, the flexible substrate, and the lead running between the microsensor device and the data acquisition device.

7. The catheter of claim 6, wherein the sensor element detects at least one of glucose concentration, pressure, temperature, pH, oxygen concentration, carbon dioxide concentration, heartbeat, fluids, the presence of chemicals and the presence of biologic materials.

8. The catheter of claim 3, wherein the flexible conduit circumscribes a tube including a distal end having an aperture therein and a proximal end having an opposed aperture therein, the tube also inhibiting fluid communication between an interior of the tube and an interior of the flexible conduit housing the microsensor device, the flexible substrate, and the lead running between the microsensor device and the data acquisition device.

9. The catheter of claim 8, wherein the sensor element detects at least one of glucose concentration, pressure, temperature, pH, oxygen concentration, carbon dioxide concentration, heartbeat, fluids, the presence of chemicals and the presence of biologic materials.

10. The catheter of claim 1, wherein the sensor element detects pressure.

11. The catheter of claim 1, further comprising a protective coating over at least the flexible substrate, the first electrically conductive material and the microsensor device.

12. The catheter of claim 11, wherein the protective coating includes a layer of a poly dimethyl siloxane (PDMS) material.

13. The catheter of claim 1, wherein the microsensor device is mounted to the flexible substrate by a flip-chip bonding process.

14. The catheter of claim 13, wherein the microsensor device is mounted to the flexible substrate by a polymer flip-chip bonding process.

15. A catheter for insertion into a body cavity, duct, or vessel for diagnostic purposes, the catheter comprising:
a flexible conduit carrying at least two microsensor devices therewith, each of which respectively adapted to generate output data from respective sensed conditions, each microsensor device being operatively coupled to a data acquisition device to communicate the output data to the data acquisition device, wherein at least the two microsensor devices are mounted to a flexible dielectric substrate carrying a lead, and wherein the flexible dielectric substrate is separate from and carried by the flexible conduit.

16. The catheter of claim 15, wherein the flexible conduit is oriented in parallel with a second conduit.

17. The catheter of claim 16, wherein the flexible conduit includes at least one orifice therein to respectively expose a sensor element of at least one of the two microsensor devices to the respective sensed condition external to the flexible conduit.

18. The catheter of claim 17, wherein the flexible conduit is circumscribed by a larger conduit having at least two openings therein to align with at least the two orifices of the flexible conduit to respectively expose at least two sensor elements of the at least two microsensor devices to the respective sensed conditions external from the larger conduit.

19. The catheter of claim 18, wherein the larger conduit circumscribes a tube oriented in parallel with the flexible conduit, the tube including a distal end having an aperture therein and a proximal end having an opposed aperture therein, the tube also inhibiting fluid communication between an interior of the tube and an interior of the flexible conduit.

20. The catheter of claim 19, wherein the interior of the flexible conduit includes the at least two microsensor devices, the flexible dielectric substrate, and the lead running between the at least two microsensor devices and the data acquisition device.

21. The catheter of claim 20, wherein at least one of the at least two sensor elements of the at least two microsensor devices detects at least one of glucose concentration, pressure, temperature, pH, oxygen concentration, carbon dioxide concentration, heartbeat, fluids, the presence of chemicals and the presence of biologic materials, and wherein another of the at least two sensor elements of the at least two microsensor devices detects at least one of glucose concentration, pressure, temperature, pH, oxygen concentration, carbon dioxide concentration, heartbeat, fluids, the presence of chemicals and the presence of biologic materials.

22. The catheter of claim 17, wherein the flexible conduit circumscribes a tube including a distal end having an aperture therein and a proximal end having an opposed aperture therein, the tube also inhibiting fluid communication between an interior of the tube and an interior of the flexible conduit housing the flexible dielectric substrate and the at least two microsensor devices.

23. The catheter of claim 22, wherein a first sensor element of the at least two microsensor devices detects at least one of glucose concentration, pressure, temperature, pH, oxygen concentration, carbon dioxide concentration, heartbeat, fluids, the presence of chemicals and the presence of biologic materials, and wherein a second sensor element of the at least two microsensor devices detects at least one of glucose concentration, pressure, temperature, pH, oxygen concentration, carbon dioxide concentration, heartbeat, fluids, the presence of chemicals and the presence of biologic materials.

24. The catheter of claim 15, wherein at least two of the microsensor devices are mounted to the flexible dielectric substrate utilizing a flip-chip bonding technique.

25. The catheter of claim 24, wherein the flip-chip bonding technique is a polymer flip-chip bonding technique.

26. The catheter of claim 15, wherein the at least two microsensor devices are proportionally spaced along at least a part of the length of the flexible conduit.

27. A method of bonding a microsensor device onto a conduit adapted for use as a component of a catheter, the method comprising the steps of:
providing a flexible dielectric substrate having a leading end;
applying a first electrically conductive material to the dielectric substrate approximate the leading end, where the first electrically conductive material includes at least one lead;
coupling the first electrically conductive material to a microsensor device to provide electrical communication between the microsensor device and the at least one lead; and
mounting the leading end of the dielectric substrate to a conduit of a catheter.

28. The method of claim 27, further comprising the step of laser cutting to separate deposits of the first electrically conductive material.

29. The method of claim 27, further comprising the steps of:
applying a photoresist material onto the flexible dielectric substrate;
selectively developing the photoresist material; and
selectively removing undeveloped photoresist material to form a cavity bounded in part by the first electrically conductive material.

30. The method of claim 27, wherein the coupling step includes the steps of:
applying a second electrically conductive material onto a portion of the first electrically conductive material; and
coupling the second electrically conductive material to a microsensor device.

31. The method of claim 30, wherein the first electrically conductive material comprises a metal or metal alloy.

32. The method of claim 27, wherein the coupling step includes flip-chip bonding of the microsensor device to the second electrically conductive material.

33. The method of claim 32, wherein the flip-chip bonding step is a polymer flip-chip bonding step.

34. The method of claim 27, further comprising the steps of:
providing a substrate carrier; and
removing the substrate carrier from the flexible dielectric substrate prior to the coupling step;
wherein the removing step is accomplished at least in part by peeling the substrate carrier from the flexible dielectric substrate.

35. The method of claim 34, wherein the substrate carrier comprises a silicon wafer.

36. The method of claim 27, wherein the coupling step includes the step of applying an alignment pedestal to the flexible dielectric substrate to align the microsensor device with respect to the flexible dielectric substrate.

37. The method of claim 27, further comprising the step of a coating at least the flexible substrate, the first electrically conductive material and the microsensor device with a protective material.

38. The method of claim 37, wherein the protective material includes a layer of a poly dimethyl siloxane (PDMS) material.

39. A multilumen catheter comprising:
a first flexible tube having a microsensor device at least partially carried thereon and operatively coupled to an output device, the microsensor device being mounted to a flexible dielectric substrate carrying a lead isolated from an environment external to the first flexible tube, the microsensor device having a sensor element exposed to the environment to detect environmental conditions in real-time and relay data evidencing such environmental conditions to the output device in real-time;
a second flexible tube including a distal end having an orifice therein and providing a conduit therein; and
a catheter receiving at least the first flexible tube and the second flexible tube.

40. The multilumen catheter of claim 39, wherein the catheter includes a wall circumscribing the first flexible tube and the second flexible tube substantially along the length of the first flexible tube and the second flexible tube.

41. The multilumen catheter of claim 39, wherein at least one of the flexible dielectric substrate and the lead is encased by the first flexible tube.

42. The multilumen catheter of claim 39, wherein the first flexible tube has a diameter of between about 0.33 mm to about 2 mm.

43. The multilumen catheter of claim 39, wherein the second flexible tube has a diameter of between about 0.33 mm to about 2 mm.

44. The multilumen catheter of claim 39, wherein the first flexible tube includes an aperture to provide the sensor element with access to the environment.

45. The multilumen catheter of claim 44, wherein the catheter wall includes an opening generally aligned with the aperture of the first flexible tube to provide the sensor element with access to environmental conditions beyond the catheter wall.

46. The multilumen catheter of claim 43, wherein:
the first flexible tube is coaxial with the second flexible tube; and
the second flexible tube includes an aperture generally aligned with the aperture of the first flexible tube to provide the sensor element with access to the external environment.

47. A multilumen catheter comprising:
a first flexible tube having a first microsensor device carried thereon, the first microsensor device being coupled to a first flexible dielectric substrate carrying a set of first conductive leads in electrical communication with the first microsensor device, the first flexible tube providing a conduit though which the first flexible dielectric substrate is received, wherein the first microsensor device includes a sensor element in communication with an environment external to the first flexible tube to detect an environmental condition in real-time and relay data evidencing such environmental condition in real-time to a remote data processing device;
a second flexible tube having a second microsensor device carried thereon, the second microsensor device being coupled to a second flexible dielectric substrate carrying a second set of conductive leads in electrical communication with the second microsensor device, the second flexible tube providing a passageway though which the second flexible dielectric substrate is received, wherein the second microsensor device includes a sensor element in communication with an environment external to the second flexible tube to detect an environmental condition external to the second flexible tube in real-time and relay data evidencing such environmental condition in real-time to the remote data processing device; and
a third flexible tube including a distal end having an orifice therein and providing a conduit for fluid flow therethrough.

48. A method of bonding a microsensor device onto a conduit adapted for use as a component of a catheter, the method comprising the steps of:
providing a flexible dielectric substrate having a leading end;
a step for forming an electrically conductive material that includes at least one lead to the dielectric substrate approximate the leading end;
a step for coupling the first electrically conductive material to a microsensor device to provide electrical communication between the microsensor device and the at least one lead; and
mounting the leading end of the dielectric substrate to a conduit of a catheter.
